(19) United States
(12) Patent Application Publication
(10) Pub. No.: US 2007/0207195 A1
(43) Pub. Date: Sep. 6, 2007

(54) Genetic Screening for Polymorphisms in Human Genes that Increase or Decrease Sensitivity to Toxic Agents

(75) Inventors: Daniel B. Yarosh, Merrick, NY (US); David A. Brown, Merrick, NY (US)

Correspondence Address: MAURICE M KLEE, 1951 BURR STREET, FAIRFIELD, CT, 06824, US

(73) Assignee: Applied Genetics Incorporated Dermatics, Freeport, NY (US)

(21) Appl. No.: 11/628,678

(22) PCT Filed: Jun. 7, 2005
(86) PCT No.: PCT/US05/20055

§ 371 Date: Dec. 6, 2006

Related U.S. Application Data
(60) Provisional application No. 60/577,822, filed on Jun. 8, 2004.
(60) Provisional application No. 60/578,530, filed on Jun. 10, 2004.

(51) Int. Cl. C12Q 1/68; A61K 9/127; G06Q 50/00

(52) U.S. Cl. 424/450; 435/6; 705/2

(57) ABSTRACT

Methods are disclosed for genetically counseling a person based on one or more polymorphisms in his or her genes that sensitize him or her to toxic agents. Methods are also disclosed for genetically screening a group of individuals and/or a human population, based on, for example, ethnicity, race, religion or geographic region, to identify individuals with such polymorphisms for counseling. The methods can be used to counsel a person who has not been genetically tested for polymorphisms but who might have increased risk for sensitivity to toxic agents due to his or her membership in a particular group and/or population. The methods use correlations between genotypes of polymorphic alleles in a panel of cell lines and sensitivity of the cell lines to toxic agents. As examples, the methods are used to identify genotypes of allelic forms of the genes TP53, OGG1, ERCC2, XRCC1, and NOS3 that increase sensitivity or resistance of cells to toxic agents.

### FIELD OF THE INVENTION

This application relates to genetic counseling and/or screening, and, in particular, relates to genetic counseling/screening with respect to toxic agents, such as, environmental toxins, food toxins, toxins administered as therapeutic agents, e.g., chemotherapy agents, exposure to ionizing radiation, e.g., x-rays, exposure to ultra-violet light, toxins generated in situ by, for example, inflammatory cells, and the like.

### LITERATURE REFERENCES

DNA Repair Gene Polymorphisms

W. Au, S. Salama, C. Sierra-Torres. Functional characterization of polymorphisms in DNA repair genes using cytogenetic challenge assays. Env. Hlth. Persp. 111:1843-1850, 2003.

L. Chen, A. Elahi, J. Pow-Sang, P. Lazarus and J. Park. Association between polymorphism of human oxoguanine glycosylase 1 and risk of prostate cancer. J. Urol. 170:2471-2474, 2003.

E-Y. Cho, A. Hildesheim, C-J. Chen, M-M. Hsu, I-H. Chen, B. Mittl, P. Levine, M-Y. Liu, J-Y. Chen, L Brinton, Y-J. Cheng, C-S. Yang. Nasopharyngeal carcinoma and genetic polymorphisms of DNA repair enzymes XRCC1 and hOGG1. Cancer Epid. Biom. Prev. 12:1100-1104, 2003.

C. Dherin, J. Radicella, M. Dizdaroglu, S. Boiteux. Excision of oxidatively damaged DNA bases by the human α-hOGG1 protein and the polymorphic α-hOGG1(Ser326Cys) protein which is frequently found in human populations. Nucl. Acids Res. 27:4001-4007, 1999.

E. Goode, C. Ulrich and J. Potter. Polymorphisms in DNA repair genes and associations with cancer risk. Cancer Epid. Biom. Prev. 11:1513-1530, 2002.

S-M. Hou, C. Ryk, A. Kannio, S. Angelini, S. Falt, F. Nyberg, K. Husgafvel-Pursianinen. Influence of Common XPD and XRCC1 variant alleles on p53 mutations in lung tumors. Environ. Mol. Mutagen. 41:37-42, 2003.

K. Janssen, K. Schlink, W. Gotte, B. Hippler, B. Kaina, F. Oesch. DNA repair activity of 8-oxoguanine DNA glycosylase I (OGG1) in human lymphocytes is not dependent on genetic polymorphism Ser³²⁶/Cys³²⁶. Mutat. Res. 486:207-216, 2001.

T. Kohno, K. Shinmura, M. Tosaka, M. Tani, S. Kim, H. Sugimura, T. Nohmi, H. Kasai, J. Yokota. Genetic polymorphisms and alternative splicing of the hOOG1 gene, that is involved in the repair of 8-hydroxyguanine in damaged DNA. Oncogene 16:3219-3225, 1998.

Y. Li, M-J. Marion, A. Rundle, P. Brandt-Rauf. A common polymorphism in XRCC1 as a biomarker of susceptibility for chemically induced genetic damage. Biomarkers 8:408-414, 2003.

B. Rybicki, D. Conti, A. Moreira, M. Cicek, G. Casey, J. Witte. DNA repair gene XRCC1 and XPD polymorphisms and risk of prostate cancer. Canc. Epid. Biom. Prev. 13:23-29, 2004.

D. Tang, S. Cho, A. Rundle, S. Chen, D. Phillips, J. Zhou, Y. Hsu, F. Schnabel, A. Estrabrook, F. Perera. Polymorphisms in the DNA repair enzyme XPD are associated with increased levels of PAH-DNA adducts in a case-control study of breast cancer. Breast Canc. Res. Treat. 75:159-166, 2002.

Y. Wang, M. Spitz, Y. Zhou, Q. Dong, S. Shete, X. Wu. From genotype to phenotype: correlating XRCC1 polymorphisms with mutagen sensitivity. DNA Repair. 2:901-908, 2003.

NOS Gene Polymorphisms

H-R. Chang, D-A. Tsao, S-R. Wang. Expression of nitric oxide synthetase in keratinocytes after UVB irradiation. Arch. Dermatol. Res. 295:293-296, 2003.

R. Fukunaga-Takenaka, K. Fukunaga, M. Tatemichi, H. Ohshima. Nitric oxide prevents UV-induced phosphorylation of the p53 tumor-suppressor protein at serine 46: a possible role in inhibition of apoptosis. Biochem. Biophys. Res. Comm. 308:966-974, 2003.

G. Ghilardi, M. Biondi, M. DeMonti, M. Bernini, O. Turri, F. Massaro, E. Guagnellini, R. Scorza. Independent risk factor for moderate to severe internal carotid artery stenosis: T786C mutation of the endothelial nitric oxide synthetase gene. Clin. Chem. 48:989-993, 2002.

G. Ghilardi, M. Biondi, F. Cecchini, M. DeMonti, E. Guagnellini, R. Scorza. Vascular invations in human breast cancer is correlated to T→786C polymorphism of NOS3 gene. Nitric Oxide 9:118-122, 2003.

G. Rossi, S. Taddei, A. Virdis, M. Cavallin, L. Ghiadoni, S. Favilla, D. Versari, I. Sudano, A. Pessina, A. Salvetti. The T-786C and Glu298Asp polymorphisms of the endothelial nitric oxide gene affect the forearm blood flow responses of Caucasian hypertensive patients. J. Am. Col. Cardiol. 41:938-945, 2003.

J. Song, Y. Yoon, K. Park, J. Park, Y. Hong, S. Hong, J. Kim. Genotype-specific influence on nitric oxide synthetase gene expression, protein concentrations, and enzyme activity in cultured human endothelial cells. Clin. Chem. 49:847-852, 2003.

X. Wang, Z. Zalcenstein, M. Oren. Nitric oxide promotes p53 nuclear retention and sensitizes neuroblastoma cells to apoptosis by ionizing radiation. Cell Death Diff. 10:468-476, 2003.

Cell Line Panel

A. Monks, D. Scudiero, P. Skehan, R. Shoemaker, K. Paull, D. Vistica, C. Hose, J. Langley, P. Cronice, M. Vaigro-Wolf, M. Gray-Goodrich, H. Campbell, M. Mayo. Feasibility of a high-flux anticancer drug screen using a diverse panel of cultured human tumor cell lines. J. Natl. Cancer Inst. 83:757-766, 1991.

Patents and Patent Applications

U.S. Pat. No. 5,077,211 (the '211 patent)

U.S. Pat. No. 5,296,231 (the '231 patent).

Patent applications WO 02080755, US2003073612, and US2002146698, and issued U.S. Pat. No. 6,291,171 (collectively, the '755 application family).

The contents of the foregoing articles, patents, and patent applications are incorporated herein by reference.

### BACKGROUND OF THE INVENTION

Cells are damaged by toxic agents that are both natural and man-made. They are damaged directly when toxic agents react with the DNA, such as in the case of ionizing or ultraviolet radiation, oxidation by reactive oxygen species, or reaction with alkylating agents. Cells may be damaged indirectly when normal metabolic processes go awry, such as when mitochondria produce excessive reactive oxygen, organelles dissolve, cells enter the apoptotic pathway, or inflammatory cells release toxic substances to combat infection. The target of these toxic events is both nuclear and mitochondrial DNA.

Toxic events can nearly always be recognized in a short time by the appearance of cell death, although this is not the only effect of toxicity. For example, solar UV radiation is lethal to cells. The cell death is recognized on the microscopic level in skin histopathology as sunburn cells, and by clinical observation as the desquamation of sheets of dead cells known as peeling. Cancer chemotherapeutic drugs are toxic to rapidly dividing cells, particularly of the gut, and this is recognized by histopathology as epithelial sloughing and by clinical observation as nausea and vomiting.

Cells respond to toxic agents with a variety of DNA repair mechanisms. These include processes that directly reverse the lesion, base excision repair processes that remove the damaged base, nucleotide excision repair processes that remove stretches of DNA, and replication bypass mechanisms that allow cell replication of damaged DNA and postpone the immediate need for repair. In human cells, DNA repair is a complex cooperation of multiple enzymes, scaffolding proteins and signaling molecules, some of which are also involved in other cell functions such as RNA transcription, cell division, and cell-cell communication. Other organisms, particularly microorganisms, manufacture small, specialized DNA repair enzymes directed to specific types of damage.

DNA repair enzymes and associated factors are controlled by the expression of genes. A mutation in the nucleotide sequence of the regulatory or structural portion of a DNA repair gene can inactivate the gene product, and this can have drastic consequences on DNA repair. For example, the genetic disease xeroderma pigmentosum is caused by mutations in DNA repair genes that interfere with the expression of one of seven genes, either by changing a critical amino acid, altering RNA splicing, truncating translation or other changes. The results are cells highly sensitive to cell killing by UV, and these patients are extremely photosensitive and have an enormously elevated rate of skin cancer.

However, surveys of genes within and between populations reveal many differences in single nucleotide bases or other small changes in nucleotide sequences which are not revealed as disease syndromes. The different gene forms which may occur at a site in the genome (a “gene locus”) are called alleles, and depending at least in part upon frequency of occurrence, these variable forms of the gene are also referred to as polymorphisms. In particular, a polymorphic allele is a site in the DNA where multiple sequences can be found in more than about 10% of a human population. The sequence can comprise one or more nucleotides and need not be a complete gene. A mutant allele, on the other hand, is the occurrence of a DNA sequence change in the genomes of about 1% or less of a human population. Again, the sequence can comprise one or more nucleotides and need not be a complete gene. A mutant allele is much more deleterious to a person than a polymorphic allele, and it is therefore eliminated from a population at a much higher rate. Alleles in the range of 1-10% frequency are considered mutant alleles if they result in a disease syndrome, and polymorphic if they do not produce overt disease.

The most frequent form of the gene in the population is the dominant allele and the less common is the variant allele. The genotype refers to the gene composition at a gene locus, and in non-sex cells there are two copies of each gene at each gene locus of each chromosome, except for the sex chromosomes of men, i.e., the X and Y chromosomes, where there is only one copy of each gene at each gene locus. A person who has one copy each of the dominant and variant alleles at a gene locus has a heterozygous genotype at that locus, while if the person has two copies of the same allele at the locus, he/she is homozygous at the locus. In sum, subject to the above exceptions, the three possible genotypes at a gene locus with a polymorphism are homozygous dominant, heterozygous, and homozygous variant. The phrase “individual genotypes” is used to identify the various genotypes actually exhibited by a group of individual human subjects at the locus, i.e., homozygous dominant, heterozygous, and homozygous variant. It is possible that in a group with a limited number of individuals one or more of the genotypes may not be exhibited.

For example, a group of say 100 individuals may only exhibit the heterozygous and homozygous dominant genotypes at a particular gene locus. The “individual genotypes” for that group would then only be homozygous dominant and heterozygous at that locus. For another gene locus, the same group may exhibit all three genotypes. For that locus, the individual genotypes would then be homozygous dominant, heterozygous, and homozygous variant.

The convention for writing a polymorphism which produces a change in an amino acid is to use the single letter in upper case representing the dominant amino acid, followed by the number representing the position in the protein, followed by the single letter representing the variant amino acid. For example, OGG1 S326C designates the polymorphism in the OGG1 gene where the dominant form has a serine at amino acid 326 while the variant form has a cysteine. In a case of a polymorphism in which the change is in a nucleotide but not in an amino acid of the protein, it is designated by the single letter in lower case for the dominant base, followed by the number representing the position of the nucleotide relative to the start of gene transcription, followed by the single letter in lower case for the variant base. For example, NOS3 t-786c designates the polymorphism of the NOS3 gene at 786 nucleotides before the transcription start where the dominant form has a thymidine and the variant form has a cytosine.

There are thousands of polymorphisms in the human population and it is not clear which ones are benign and which ones have subtle effects. The existence of a variant polymorphism in a gene is not proof that it has any consequence. In addition, some variant polymorphic alleles actually confer increased activity or benefit, so that a variant polymorphism is not proof of a gene defect.

Many polymorphisms have been described in DNA repair genes. In some cases their potential link to cancer has been examined (Goode et al., 2002). There are only a few cases in which a preponderance of evidence indicates a relationship between a particular polymorphism and cancer risk. Most often, the studies are small and not well controlled, the effects are small and the reports are contradictory. In addition, the effect of the polymorphism on gene expression or the activity of the gene product has not been demonstrated, and it is not known how the polymorphism results in increased cancer risk. Further, the polymorphisms are not linked to other potential effects, such as cell death or aging.

The problem in the present art is illustrated in the human gene for 8-oxo-guanine glycosylase 1 (OGG1). This gene encodes a DNA glycosylase that participates in base excision repair of oxidized guanine bases. The OGG1 polymorphism S326C has been associated with an increased risk of several types of cancer (Goode et al., 2002). However, three separate biochemical studies of the activity of the protein produced by the variant gene failed to identify any deficit in activity or reduced DNA repair of oxidatively damaged DNA (Kohno et al., 1998; Dherin et al., 1999; Janssen et al., 2001). It remains a mystery, therefore, whether or not the polymorphism in OGG1, or some linked and hidden allele, or something else, is responsible for the cancer risk.

Other molecules that contribute to the response to toxic agents are signaling molecules. The molecular signal molecule nitric oxide (NO) induces vasodilation, and is made by the enzyme nitric oxide synthetase (NOS) from L-arginine. Polymorphisms in the constitutive nitric oxide synthetase NOS3 gene have been described, one of which (t-786c) in the promoter region reduces NOS3 gene expression, protein concentrations and enzyme activity (Song et al., 2003). The cc homozygous variant genotype has been linked to diseases such as internal carotid artery stenosis (Ghilardi et al., 2002) and primary essential hypertension (Rossi et al., 2003). The homozygous variant genotype was also linked to a reduction in breast cancer invasion, presumably because reduced NO production resulted in reduced vasodilation and therefore reduced opportunity for invasion (Ghilardi et al., 2003). The toxic agent UV radiation is known to induce NO production in skin cells, but this has been connected with increased cytotoxicity (Chang et al., 2003). NO induced after ionizing radiation has also been reported to increase apoptosis and inhibit proliferation (Wang et al. 2003). On the other hand, UV-induced NO has been reported to inhibit apoptosis and thereby reduce cytotoxicity (Fukunaga-Takenaka et al., 2003). In addition, these radiation studies all concern NO induced by NOS2, or iNOS, and not constitutive NO produced by NOS3 (eNOS). In view of all this contradictory prior art, a practitioner would not at all be clear as to what effect a NOS3 polymorphism might have on cytotoxic responses.

DePhillipo and Ricciardi, in the '755 application family, purport to describe a method of assessing sensitivity to oxidative stress by polymorphism analysis, but their methods are without scientific support and teach away from the present invention. They direct their invention to “disorder-associated polymorphisms” (paragraph 28) without listing specific correlations between disorders and polymorphism genotypes. In point of fact, in a large proportion of cases of polymorphisms there are conflicting reports of correlations with human disease or pathological state. They do not describe how to resolve conflicting data in determining if a polymorphism is indeed disorder-associated and therefore useful, or if it is not associated (neutral) and therefore not useful. Further, the application is silent on whether the disease-associated polymorphism must be homozygous or can be heterozygous, although this is central to the scientific literature. The present invention describes in detail how to determine which polymorphic genotypes increase or decrease cytotoxicity.

DePhillipo and Ricciardi assert (paragraph 11) that “occurrence of any of the polymorphisms is an indication that the human is more susceptible to oxidative damage”. As shown in the examples set forth below, this plainly teaches away from the present invention in that some polymorphisms, such as the XRCC3 R399Q polymorphism, increase resistance to cytotoxic agents and thereby reduce the risk of disease. In another case, the TP53 heterozygous genotype contains both the dominant and variant allele and is sensitive relative to the homozygous variant genotype, a situation which is not even considered by the '755 application. Further, the NOS3 t-786c polymorphism increases the risk of arterial disease but reduces the risk of invasive breast cancer, making it impossible for the practitioner to know which form of the gene is indeed “disorder-associated”. Therefore the '755 application provides incomplete, incorrect and conflicting instructions on how a practitioner should treat a disorder-associated polymorphism even if he/she were able to identify one. The present invention describes specifically how to determine which polymorphic genotypes increase sensitivity to cytotoxicity and therefore what recommendations to make to a person with such a polymorphic genotype.

In specific, the '755 application asserts (paragraph 44) that “numerous genes encode components of the human DNA repair system, and disorder-associated polymorphisms in substantially any of these genes can be informative of the susceptibility of the individual to oxidative stress.” This teaches away from the present invention that in some DNA repair genes, the homozygous variant genotype increases and sometimes decrease resistance, in other genes the homozygous dominant and homozygous variant genotypes are more sensitive than the heterozygous genotype, and in other DNA repair gene polymorphisms associated with disorders, such as the XRCC1 R194W polymorphism, there is no difference between the sensitivity of the homozygous dominant and heterozygous genotypes.

For all these reasons, implementation of the practice described in the '755 application would not lead to the results of the present invention, and would be no better than classifying the sensitivity of people without any knowledge of their polymorphism status.

### SUMMARY OF THE INVENTION

In certain of its aspects, the present invention provides genetic counseling methods whereby a biological sample is obtained from a person, and the person's genotype is determined at a gene locus that has polymorphisms in the population. The person's genotype is then compared to a correlation between the sensitivity of cell lines with polymorphisms at that locus and growth inhibition by toxic agents. The correlation is then used to recommend a therapeutic regimen, a change in behavior (e.g., a change in diet, a reduction in exposure to UV light, or the like), or a cosmetic application. For example, the present invention identifies polymorphic forms of the genes TP53, OGG1, ERCC2, XRCC1 and NOS3 that increase the sensitivity or resistance of a person to toxic agents. Further, the present invention may be used to screen a group of individuals for polymorphisms, for the purpose of advising a person with a sensitizing polymorphism, or advising a person in the same group or in a corresponding group who was not tested that he or she may be sensitive because of the membership in, or commonality with, the group.

The invention further provides a method for identifying polymorphisms that confer hypersensitivity to cytotoxic drugs by determining the polymorphic genotype of genes in a panel of cells and correlating the genotypes with the response of the panel of cells to cytotoxic agents.

Additional features and advantages of the invention are set forth in the detailed description which follows, and in part will be readily apparent to those skilled in the art from that description or recognized by practicing the invention as described herein.

It is to be understood that both the foregoing general description and the following detailed description are merely exemplary of the invention, and are intended to provide an overview or framework for understanding the nature and character of the invention as it is claimed. The accompanying drawing is included to provide a further understanding of the invention, and is incorporated in and constitutes a part of this specification.

### BRIEF DESCRIPTION OF THE DRAWING

FIG. 1 shows the relative sensitivity to cytotoxicity of the genotypes of the polymorphism at the OGG1 S326C locus. On the left, the percent resistance is the average of the sensitivity of the heterozygous or homozygous variant genotype divided by the average sensitivity of the homozygous dominant genotype. Thus the homozygous dominant genotype is set at 100%. More resistant genotypes are>100% and less resistant genotypes are<100%. On the right, the figure shows the percent of tested drugs for which the heterozygous or homozygous variant genotype is more sensitive than the homozygous dominant genotype. The homozygous dominant genotype is arbitrarily set at 50% since if it and another genotype had equal sensitivities to the toxic agent(s) then they would have equal chances of being scored as more resistant. More resistant genotypes are<50% and more sensitive genotypes are>50%.

### DETAILED DESCRIPTION OF THE INVENTION AND ITS PREFERRED EMBODIMENTS

As discussed above, the present invention identifies genes whose polymorphic genotypes either increase or decrease sensitivity to cytotoxic agents. As shown in Example 2, the TP53 P72R polymorphism results in a heterozygous genotype that is hypersensitive to cytotoxicity relative to the homozygous dominant or homozygous variant forms. As shown in Example 3, the OGG1 S326C polymorphism results in a heterozygous genotype that is relatively resistant to cytotoxicity and a homozygous variant genotype that is most sensitive. As shown in Example 4, the ERCC2 D312N results in a homozygous variant genotype that is more sensitive to cell killing than the homozygous dominant and heterozygous genotypes. As shown in Example 5, the XRCC1 R399W polymorphism results in a homozygous variant genotype which is most resistant and a homozygous dominant genotype which is most sensitive to cytotoxicity. As shown in Example 6, the NOS3 t-786c polymorphism results in a homozygous variant genotype that is most sensitive to cytotoxicity. Not all polymorphisms described in the literature confer sensitivity or resistance to cytotoxic agents, and not all polymorphisms that have been related to diseases or disorders confer sensitivity or resistance to cytotoxic agents. The allele frequencies that are presently described in human populations are taken from the Utah polymorphism project, (www.genome.utah.edu/genesnps), which cites various specific anonymous and ethnic or geographic databases, such as PDR90, TSC 42 AA, CAUC1.

The frequency of a polymorphic allele will vary among subsets and racial groups of the human population, and in the extreme case a dominant allele in one group may be the variant allele in another group. As an example, the dominant allele for hair color among African Americans is black, while the dominant allele for hair color among Swedes is blonde. Therefore, whenever known, the subset or racial group of the group of individuals under study should be specified in describing the polymorphism. Among some subsets of the human population, a homozygous variant genotype may be so rare that a large number of samples must be collected in order to obtain a fair representation of the genotype. If such a sufficiently large group is not achieved, erroneous results may be obtained, either ignoring a significant effect or attributing one where none exists. For example, in the group of cell lines listed in Example 1, no homozygous variant genotypes were found at the PTGS2 gene V511A site, only 4 homozygous variant genotypes were found at the MGMT gene I143V site, and only 2 homozygous variant genotypes were found at the TNFα c-863a site. Many methods of statistical analysis may be used to evaluate the validity of the data and conclusions, and no one method may be appropriate for all cases. Based on the present disclosure, one skilled in the art will be able to select a method with the least data manipulation whose assumptions are consistent with the data and which achieves objective confidence that a result will be reproducible by other equally valid studies. An example of such a method of statistical analysis is shown in Example 1. In the preferred embodiments of the invention, the null hypothesis is rejected only when the statistical significance level (p-value) is less than or equal to 0.05.

The genotypes may be detected in any sample from a person that contains sufficient DNA from both copies of the gene containing the polymorphism of interest, or RNA or protein expressed from both genes. The exception is a polymorphism in a gene located on the X or Y chromosome, which are hemizygous in men. The sample may be collected from a living or dead person, and it may be collected by swabbing, scraping, cutting, biopsy or other means of extracting tissue. It may also be collected from bleedings, secretions, excretions, lacrimations, perspiration, expectorations, ejaculations or other emissions from the body, and it may be collected from placenta, amniotic fluid and cells therein, or other tissue related to a fetus. In the case of samples from sex cells, either egg or sperm, each of which contains only the haplotype or one copy of the genes, a sufficiently large sample must be used to ensure that it represents both copies of the gene in question. In a preferred embodiment, the sample is collected by scraping the buccal mucosa and analyzing the cells so recovered.

The polymorphism genotype may be determined by any method that determines the DNA sequence and, if appropriate, the coding sequence at the polymorphism site of both copies of the gene. The polymorphism may in some cases be detected by analyzing RNA or protein produced from the gene containing the polymorphism, and thereby deducing the DNA sequence. The DNA sequence may be determined from purified or partially purified DNA which has been treated by any method that selectively recovers the DNA without destroying its integrity, such as phenol extraction and ethanol precipitation. The DNA sequence may be analyzed by many methods, including polymerase chain reaction (PCR) primer extension, probe hybridization, chemical sequencing, dideoxy sequencing, gel electrophoresis of single stranded nucleic acid, or other methods. In a preferred embodiment, the DNA is purified from cells by phenol extraction and ethanol precipitation, and analyzed using PCR primer extension.

The information concerning one or more polymorphisms within an individual or a group of individuals that affects cellular cytotoxicity may result in changes in medical treatment, personal care, diet, or behavior of individuals, families, groups of individuals, or populations. The information may change decisions in commerce, such as what and how to provide insurance or other financial services to people, and what products or services to offer and to whom. The information may change the flow of other information, either by increasing the amount of information, for example, information which is available to describe a person, group of persons, or population, or by causing a restriction in the flow of information, for example, by separating individual or group identifiers from information available to describe a person, group of persons, or population. The information may change government or public policies and laws regarding public health, sanitation, public works, environmental pollution and remediation, for example by recommending that public health or pollution standards be increased in areas where a particular ethnic population harboring a high incidence of a particular genotype makes them more susceptible to environmental toxins. In a preferred embodiment, the information is used to direct an individual to use a particular product, e.g., a therapeutic and/or cosmetic product. In a preferred embodiment, the product contains a DNA repair enzyme. In another preferred embodiment, the product contains antioxidants and vitamins.

Treatment of an individual who has one or more polymorphisms that alter cytotoxicity may include deletion, repair or replacement of the target polymorphism in one or several of the cells of the body, for example, by means of gene therapy whereby a gene is delivered to the target tissue by means of a virus or other vector. It may include treatment with DNA or other cellular repair enzymes, antioxidants, drugs, foods, chemicals or other substances or gases, e.g., nitric oxide. It may include modification of behavior for prevention or protection against an insult, diet change or nutritional therapy, psychological or psychiatric counseling, aroma therapy, aural therapy, visual therapy or physical therapy.

In the case of treatments employing products, the active ingredient or ingredients may be formulated in any suitable cosmetic or pharmaceutical carrier. For topical application, this can include topical creams, lotions, serums, milks, emulsions, gels, shampoos, hair rinses, solid forms, powders, waxes and two- or multiple component mixing systems for application to the face, neck, arms or hands, legs or feet, trunk, abdomen, hair, scalp or mucus membranes. The treatment can also be formulated for intravenous, subcutaneous, intramuscular or intraperitoneal injection or other forms of injection administration. It may also be formulated in aerosols for administration by nasal spray to the nose or inhalation therapy to the lungs. It may also be formulated in drop or wash form for application to the eyes or ears. The treatment may be formulated in suppository or swab for application to the rectum. The treatment may also be formulated for oral intake in the form of pills, gel caps, capsules, or formulated into food supplements such as solid food, drinks or slurries. In a preferred embodiment the treatment is formulated in the form of a topical treatment.

The treatment may be used to treat a fetus in utero, a premature baby, a newborn, an infant, a child, an adult, and/or an elderly person. The treatment may be used to treat a healthy person, who is not experiencing any symptoms from increased or decreased cytotoxicity, or it may be used to treat a person who is already suffering from a symptom or a disease related to cytotoxicity. The treatment may be in the form of an increased dosage of a product that is already in use, or it may be a new product designated specifically for a person with a particular genotype. In a preferred embodiment, the treatment is a product not previously used in children, and is used beginning in childhood among those who have no symptoms related to the genotype. In another preferred embodiment, the dosage and use frequency of a product is increased as a person increases in age through adulthood.

The effects of the treatment may be observed immediately or over a few hours after use, such as avoidance or reduction of erythema, irritation, nausea, vomiting, fever or sunburn. It may be observed over the course of a few days, such as by the avoidance or reduction of epithelial desquamation, sloughing or peeling, inflammation, mucous secretion, or general or antigen specific suppression of the immune system. The effects may also be observed over the course of several months or years, such as the prevention or reduction of (i) the signs and symptoms of aging, including wrinkles, cataracts, loss of eyesight, hearing or other senses; (ii) the appearance of pre-malignancies or benign tumors, for example of the skin or colon; and (iii) chronic diseases associated with aging including heart disease, lung disease, arthritis or cancer. In a preferred embodiment, the treatment reduces the immediate effects of sunburn, the intermediate effects of inflammation, peeling and suppression of the immune system, and the long term effects of wrinkling and skin cancer.

In the specific case of topical treatment with a DNA repair enzyme, the topical formulation may include any of a number of DNA repair enzymes or mixtures thereof, now known or subsequently discovered or developed, such as, photolyase, T4 endonuclease V, UV endonuclease from Micrococcus luteus, 8-oxo-guanine glycosylase 1 (OGG1), and/or O⁶-alkylguanine-DNA alkyltransferase. The enzyme may be prepared for delivery to the skin in any number of ways, including liposomes, non-phospholipid vesicles, non-lipid capsules, trapped within an inert matrix or suffusing a porous matrix. The enzyme may be delivered to the skin and reach living cells due to the properties of the enzyme itself, the delivery vehicle, or by methods to permeabilize the skin such as iontophoresis or chemical treatment. In a preferred embodiment, the DNA repair enzyme is selected from the group of photolyase, T4 endonuclease V, OGG1, and mixtures thereof, and is encapsulated in liposomes as described in the '211 and '231 patents.

Treatment with antioxidants or vitamins may include any number of purified compounds, salts, pro-antioxidants or pro-vitamins or their mixtures or extracts of biomass that contain these compounds. The antioxidants and vitamins may be well-known, such as vitamin A, vitamin C or vitamin E, less well known such as L-ergothioneine and resveratrol, or antioxidants and vitamins yet to be discovered. The antioxidants and vitamins may be delivered orally or topically or both, or by any other suitable method of delivery. In a preferred embodiment, the topical product contains vitamin C with vitamin E or vitamin C with L-ergothioneine.

The invention provides a method for determining whether or not a polymorphic allele increases or decreases a person's sensitivity to toxic agents by comparing his or her genome to the genomes of a panel of cell lines that have been tested with toxic agents. The cell lines included in the panel should include sufficient representation of the polymorphic alleles in the group of individuals or population that is to be counseled so as to produce a statistically valid result. This means that fewer than 10 cell lines in the panel is inadequate, and more than 40 is best, with 25 generally being the minimum number for reliably obtaining valid results. The cell lines should be robust and easily cultured, so that measurements of growth inhibition are significant and reproducible. The panel should not contain any lines with known mutations that affect cytotoxicity. If such lines are included, statistical methods should be used, such as testing models with the mutation status as a covariate, to discount or eliminate the effect of the mutation on the outcome. For example, in the examples presented below, the occurrence of mutations in the TP53 mRNA was not found to be a significant covariate of the polymorphic alleles.

Each cell line should be a homogenous culture so that the determination of the genotype is unambiguous. Immortalized fibroblast, lymphoblasts or tumor cell lines are good candidates for a panel. An example of such a cell line panel used for growth inhibition screening is the panel maintained by the National Cancer Institute and used below in the examples (Monks et al., 1991).

Tests for growth inhibition by toxic agents are well established in the art. Many methods are available for determining cytotoxicity, such as colony forming ability, trypan blue exclusion, MTT assay and histological staining. Toxic agents are tested over a wide range of concentrations, in order to accurately determine, for example, a GI(50) value (see below). Sulfohodamine B staining methods are well suited to robotic automation of high-throughput screening of many cell lines, which are tested against many toxic agents and at many concentrations. The standard method used in the examples presented below was described by Monks et al., 1991. The method tests a toxic agent over five 10-fold dilutions in cells for 48 hrs in a 5% CO₂ atmosphere and 100% humidity. For most agents the highest concentration was 0.1 mM, but for the standard agents the highest well concentration depended on experience with the agent.

The method may be used to correlate polymorphic alleles that affect sensitivity to a broad spectrum of toxic agents or to a particular cytotoxic agent or class of agents. Testing with a broad spectrum of toxic agents is preferred since it can simulate toxic exposure from a variety of sources, e.g., environmental sources, over a long time period, and can provide correlations between GI(50) and polymorphic alleles that can apply to lifetime human exposure to both known and unknown toxins. Testing with a particular toxic agent or class of agents provides correlations between GI(50) and polymorphic alleles that apply to special conditions of human exposure, such as exposure to intense sunlight, ionizing radiation or a specific chemical contamination. In the examples presented below, 40 toxic cancer chemotherapeutic drugs were used to treat the cell lines. As noted in Example 1, these drugs have several modes of action, including alkylation, anti-mitotic, anti-metabolites, topoisomerase I or II inhibitors and DNA/RNA inhibitors. In addition to their primary mode of action, these toxic drugs induce secondary reactions within cells that include the release of toxic reactive oxygen species. Therefore, this array of drugs models lifetime exposure to toxins.

The polymorphisms that are selected for correlation with the growth inhibition in the cell line panel are preferably those most likely to affect the response of humans to toxic agents. These include polymorphisms in DNA repair genes whose deficits cause sensitivity to extracellular insult, including genes related to base excision repair, nucleotide excision repair, photoreactivation, or alkyltransferase. The products of these genes may include glycosylases, endonucleases, exonucleases, ligases, helicases, and scaffolding proteins. Further, the polymorphisms selected for correlation may also be in genes that code for enzymes or products that participate in cytotoxic responses, such as, apoptosis, or signaling responses that trigger cytotoxic responses, such as, cytokines, cyclic nucleosides or nitric oxide, such that alterations in these genes increase or decrease toxicity. Other polymorphisms may be selected for correlation because they are associated with a disease by epidemiology. Finally, remaining polymorphic alleles may be determined in each of the cell lines in the panel by high-throughput screening for some or all remaining polymorphisms, and correlating each polymorphic genotype with the toxic response of the cell lines. The polymorphisms in genes may be known today or discovered in the future. In a preferred embodiment, the method is used to discover polymorphisms in DNA repair and signaling genes that alter cellular response to cytotoxic agents.

The polymorphisms selected for correlation may be in a coding or noncoding region, in a promoter or regulatory sequence, or in non-transcribed DNA, or in any part of the genome. The polymorphism can be a single base change, an insertion or deletion, inversion, rearrangement or any other change in the nucleotide sequence of the DNA. The method can be used to correlate sensitivity to toxic agents with single polymorphisms or haplotypes (combinations of polymorphic alleles). In a preferred embodiment, the invention is used to correlate single nucleotide polymorphisms in either coding or regulatory sequences with sensitivity to toxic agents.

Without intending to restrict in any way the scope of the invention, the following examples are presented to illustrate various of the invention's aspects and its use.

### EXAMPLE 1

### Correlation of Genotype with Cytotoxic Phenotype

Single nucleotide polymorphisms (SNPs) in the genes under investigation were detected by the Amplifour™ system (Marligen, Gaithersburg Md.), which is a PCR-based detection method using two different fluorescent primers for the dominant or variant alleles with fluorescence detection. The primers are labeled with either fluorescein (green) or sulforhodamine (red) and generate a fluorescence signal of the respective color upon incorporation into a PCR product. Incorporation of only one primer indicates either homozygous dominant with one color, or homozygous variant alleles with the other color, while incorporation of both primers indicates the heterozygous genotype with both colors.

The cell lines were from the National Cancer Institute (NCI) tumor cell screen and are listed in Table 1. DNA purified from each cell line was analyzed for SNPs at the alleles of interest.

Each cell line was tested with the drugs listed in Table 2, except where indicated in that table. Cell suspensions were added to a microtiter plate and incubated for 24 h at 37° C. The drugs were added at concentrations spanning five 10-fold dilutions, and incubated for 48 h. The cells were assayed by staining the cells with sulforhodamine B, and a plate reader was used to read the optical densities. The results are expressed as a GI50 value, which is the drug concentration producing 50% growth inhibition, with correction for the cell count at time zero.

In describing the response of the polymorphic genotypes to these drugs, relative sensitivity means that a particular genotype had a lower average GI(50) than the other genotypes, and relative resistance means that a particular genotype had a higher average GI(50) than the other genotypes.

The drug sensitivities for the polymorphs of each gene of interest were calculated in the following manner. For each drug, the average GI(50) for each polymorphic genotype was calculated, e.g., the GI(50) for the homozygous dominant, heterozygous and homozygous variant genotypes. Then the GI(50)s for all the drugs were analyzed among genotypes by nonparametric Friedman Repeated Measures ANOVA. In this test the GI(50) is only used for ranking among the genotypes for each drug, so errors due to sample size among genotypes are minimized. Statistical analysis of each pair of polymorphic genotypes was done by the Tukey-Kramer Multiple Comparison Test. Analysis of relative sensitivity of the genotypes to an individual drug was by parametric ANOVA.

To determine the relative sensitivity of each pair of genotypes, the relative sensitivity to each drug was calculated by dividing the GI(50) of one genotype by the GI(50) of the other. The relative sensitivities for all the drugs were then averaged. For each pair of genotypes, we also calculated the percent of drugs in which one genotype was more sensitive than the other. These two measurements, average relative sensitivity and percent of drug sensitivity, are measures of the depth and breadth, respectively, of the difference between two genotypes.

### EXAMPLE 2

### Gene TP53 Polymorphism P72R

The TP53 gene codes for a protein that is important in transcriptional regulation of the cellular response to DNA damage, and in fact has been called the “Guardian of the Genome.” Mutations in this gene increase the risk of cancer, and the gene is therefore a tumor suppressor gene. The polymorphism at position 72 is a change from proline to arginine. The frequency of the TP53 variant allele among the cell lines was 32% while the frequency in the Caucasian and African American population is 27%. The distribution of TP53 P72R polymorphisms among the cell lines did not follow the Hardy-Weinberg distribution. We would expect the frequency of the heterozygous genotype to be greater than the homozygous variant genotype. However, the homozygous dominant genotype was 61%, the homozygous variant was 25% and the heterozygous genotype was 14%. The deviation from the expected distribution, assuming a 27% variant gene frequency, was statistically significant (p=0.0014, Chi-square test). This reflects depletion in the heterozygous genotype among the cell lines.

Overall, there was a statistically significant difference among the genotypes in resistance to the drug panel (Friedman ANOVA, p<0.001). The order of resistance was homozygous variant>homozygous dominant>heterozygous genotype. The difference between heterozygous and either the homozygous dominant or homozygous variant genotype was significant (p<0.001, Tukey-Kramer test), but the difference between the homozygous dominant and homozygous variant genotype was not (p>0.05, Tukey-Kramer test). On average, the heterozygous genotype had 73% of the resistance of the homozygous dominant genotype, and was more sensitive in 85% of the drugs tested. The heterozygous genotype had 48% of the resistance of the homozygous variant genotype, and was more sensitive in 87% of the cases.

Taken together, the results demonstrate that the heterozygous genotype confers relative sensitivity to growth inhibition or cell death over a wide range of cytotoxic challenges compared to the homozygous dominant or homozygous variant genotype. This indicates that those people with the heterozygous genotype should take additional safety precautions, such as minimizing exposure to sunlight, ionizing radiation or air and water pollution, using sunscreens more often or of higher SPF rating than normal, and consuming antioxidants in higher amounts or more often than normal.

### EXAMPLE 3

### Gene OGG1 Polymorphism S326C

The OGG1 gene codes for the 8-oxo-guanine glycosylase, which is a DNA repair gene that recognizes 8-oxo- or 8-hydroxy-guanine, and other related oxidized bases, in DNA, and makes a single-stranded break in DNA at the site of the damaged base. 8-oxo-guanine is the most common DNA lesion produced by oxidation and its level in the urine has been used as a biomarker for oxidative damage to the animal.

The normal allele at position 326 is serine. The frequency of the variant cysteine allele in the general population varies with racial grouping: 10% in African Americans, 20% in Caucasians and Hispanics, and 38% in Pacific Rim peoples. In the cell panel the variant allele frequency is 28% of the panel, similar to Caucasian and Hispanic populations. The heterozygous genotype was 32% of the population, while the homozygous dominant and homozygous variant genotypes were 68% of the population.

The sensitivities of the genotypes to all the drugs differed (p=0.0005, Friedman ANOVA). The order of resistance was heterozygous>homozygous dominant>homozygous variant. In a post-test statistical analysis using the Tukey-Kramer test, the homozygous variant genotype was significantly different than either the homozygous dominant or heterozygous genotype (p<0.01, Tukey-Kramer test), but in this test the heterozygous and homozygous dominant were not statistically significantly different, although the q-statistic was borderline significant. By the nonparametric Dunn's multiple comparison test, the heterozygous genotype was statistically significantly more resistant than the homozygous dominant genotype.

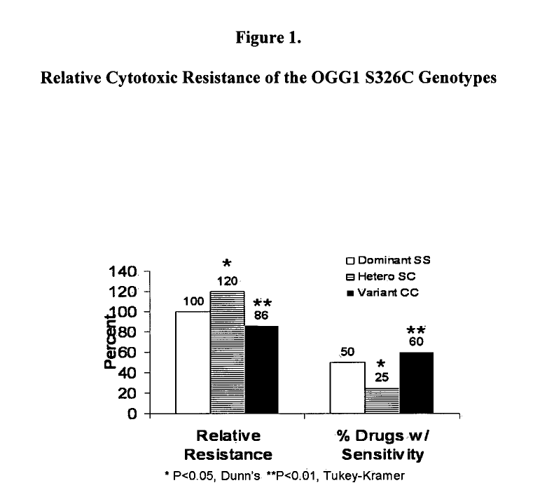

The heightened sensitivity of the homozygous variant genotype is presented graphically in FIG. 1. This shows that the homozygous variant genotype was more sensitive than the homozygous dominant genotype (only 86% relative resistance), and was more sensitive than the homozygous dominant genotype in 60% of the drugs, and these differences were statistically significant. If the homozygous dominant genotype and the homozygous variant genotype had equal sensitivities on average to the toxic agent(s), then instead of the 60% shown in the right hand part of FIG. 1 for the homozygous variant genotype, one would have seen 50%, i.e., the expected result from random fluctuations.

FIG. 1 is consistent with the observed increased risk of the variant allele for prostate cancer (Chen et al., 2003), nasopharyngeal cancer (Cho et al., 2003), and esophageal, lung and stomach cancer (Goode et al., 2002). In the case of prostate or stomach cancer, the proximal cause of the cancer is not well known, while in non-smokers the cause of the other cancers are also not well understood. Therefore, the present art provides no clear guidance to those with the homozygous variant genotype. However, according to the present invention, those people with the homozygous variant genotype should take additional safety precautions, such as minimizing more than normal the exposure to oxidative damage such as air pollution, overly cooked foods, sunlight, excessive temperatures, or ionizing radiation. They should also consume antioxidants at higher levels or more often, and use topical products that enhance the activity of the OGG1 glycosylase and other DNA repair pathways.

The homozygous variant genotype had only 76% the resistance of the heterozygous genotype, and was more sensitive in 73% of the drugs. The homozygous dominant genotype had only 83% of the resistance of the heterozygous genotype, and was more sensitive in 75% of the cases. This suggests that the heterozygous population is more resistant to environmental damage. The increased resistance of the heterozygous genotype has not been previously described, and would never be detected in biochemical assays in which purified protein produced from either one or the other alleles was tested alone. There is no indication that those with the heterozygous genotype need to take the extra precautions necessary for those with the homozygous dominant or variant genotypes.

### EXAMPLE 4

### Gene ERCC2 Polymorphism D312N

The ERCC2 gene, also known as the XPD gene, codes for a subunit of the transcription factor TFIIH, which is involved in DNA unwinding during the nucleotide excision type of DNA repair and also initiation of basal transcription. Patients defective in this factor have the genetic disease xeroderma pigmentosum of the complementation group D type.

The normal allele at position 326 codes for aspartic acid and the variant allele codes for asparagine. The frequency of the variant allele in the general population is 24%. In the cell panel the variant allele frequency is 30%. The homozygous dominant genotype was 58%, the heterozygous genotype was 25%, while the homozygous variant genotype was 18% of the cell line population.

The sensitivities of the genotypes to all the drugs differed (p=0.0003, Friedman ANOVA). The order of resistance was homozygous dominant>homozygous variant=heterozygous. In post-test analysis using the Tukey-Kramer test, the homozygous dominant genotype was significantly different than either the heterozygous (p<0.01, Tukey Kramer test) or homozygous variant genotypes (p<0.05, Tukey-Kramer test), but the heterozygous and homozygous variant genotypes were not statistically significantly different. Thus, overall, the homozygous dominant genotype was significantly more resistant than either of the other two genotypes.

Although the degree of resistance of the homozygous dominant group was statistically significant it was small. The homozygous variant genotype had 91% the resistance of the homozygous dominant genotype, and was more sensitive in 70% of the drugs. The heterozygous genotype had 90% of the resistance of the homozygous dominant genotype, and was more sensitive in 83% of the cases. This suggests that the homozygous dominant population is about 10% more resistant to environmental damage than the other genotypes.

The increased resistance of the homozygous dominant genotype to cell killing has not been previously described. Contradictory findings have been reported for the relationship of the homozygous dominant genotype to lung cancer (Goode et al., 2002). The variant allele was not related to basal cell carcinoma risk overall, but only in those with a family history of it (Goode et al., 2002). The variant allele has also been related to the risk of prostate cancer (Rybicki et al., 2004). The variant allele was not found to be related to the risk for breast cancer (Tang et al., 2002). The variant allele was also not found to be related to the levels of polycyclic-aromatic hydrocarbon adducts to DNA in normal or benign breast tissue, although it was related to the levels in tumor tissue (Tang et al., 2002). This finding in the prior art teaches away from the present invention in that it demonstrates that the variant polymorphism in normal people does not predict DNA adduct levels in breast tissue. The finding in tumor tissue is clinically uninformative since it indicates the adduct level difference is secondary to the onset of the cancer. Others have found that the variant allele is associated with chromosome changes after exposure to some toxic agents, like UV, but not others, like x-rays (Au et al., 2003). These chromosome changes are predominantly associated with cancer risk, and not cell survival.

The procedures of the present invention indicate that those with the homozygous variant or heterozygous genotype need not be overly concerned with their relative sensitivity, but that they may benefit mildly from avoiding exposure to environmental toxins, consuming more antioxidants, and applying products that enhance DNA repair.

### EXAMPLE 5

### Gene XRCC1 Polymorphisms R194W and R399Q

The XRCC1 gene encodes a DNA base excision repair protein that functions in the correction of single-stranded breaks. Single-stranded breaks are commonly formed by spontaneous damage, ionizing radiation and alkylating agents. XRCC1 serves as a scaffolding protein to coordinate the activity of catalytic enzymes to repair the break.

One polymorphism is R194W, where an arginine is replaced in the polymorphic form by a tryptophan at amino acid 194. No homozygous variant genotypes were found in the cell line panel. The sensitivity of the homozygous dominant and heterozygous genotypes to the toxic agents did not differ (p=0.669, Wilcoxon matched-pairs signed-rank test), and the heterozygous genotype was sensitive to only 3% more drugs than the homozygous dominant, but this difference was not statistically significant. These genotypes are not correlated with sensitivity to toxic agents.

Another polymorphism occurs at position 399, where the dominant allele is arginine. The frequency of the variant glycine allele at this position is 47% for Caucasians, 46% for Pacific Rim people, 33% for Hispanics and as low as 10% among African Americans. Within the cell panel the allele frequency was 36%, and the frequency of the homozygous variant genotype was 21%. The frequencies of the homozygous dominant and heterozygous genotypes were 49% and 30%, respectively.

The differences among the groups in sensitivity to all the drugs were significant (p<0.0001, Friedman ANOVA). The order of resistance was homozygous variant>heterozygous>homozygous dominant, and the homozygous variant was statistically significantly more resistant than either of the other genotypes (p<0.01, Tukey-Kramer test). The difference was also significant in analysis of sensitivity to the single drug vinblastine (p=0.025, ANOVA), and in post-tests of this drug the homozygous variant genotype was significantly more resistant than the homozygous dominant genotype (p=0.045, Bonferroni adjusted multiple comparison test). This finding is unexpected since vinblastine is an anti-mitotic and not normally associated with single-strand breaks.

The homozygous variant genotype increased drug resistance by 35% relative to the homozygous dominant genotype, and the homozygous variant genotype was more resistant than the homozygous dominant genotype to 82% of the drugs. The homozygous variant genotype increased drug resistance by 29% relative to the heterozygous genotype, and the homozygous variant genotype was more resistant than the heterozygous genotype to 65% of the drugs. The difference between homozygous variant and homozygous dominant genotypes was significant, but the difference with the heterozygous genotype was not. The difference between the heterozygous and homozygous dominant genotype was also significant.

These results are consistent with the findings that the variant allele is associated with a decreased risk for nonmelanoma skin cancer, esophageal cancer and bladder cancer (Goode et al., 2002). For those with fewer than three sunburns, the homozygous variant genotype was protective for nonmelanoma skin cancer, but carried an increased risk for those with more than three. Other studies have given conflicting results in squamous cell carcinoma of the head and neck and lung cancer (Goode et al., 2002).

For nasopharyngeal cancer, no association has been reported (Cho et al., 2003). Cellular or biochemical assays have given conflicting results. The homozygous variant genotype did not affect transversion mutations in the p53 gene, nor repair of UV damage, nor strand break repair, nor cell survival after alkylation damage (summarized in Hou et al., 2003). Unexpectedly, higher levels of vinyl chlorine adducts were found in the homozygous variant genotype than the others (Li et al., 2003), and more breaks per cell were found after bleomycin treatment in homozygous variant genotypes than the others, although there was no difference after BPDE treatment (Wang et al., 2003).

These results suggest that those people with the homozygous dominant or heterozygous genotypes would benefit from minimizing their exposure to toxic agents, and using products that counteract toxic damage at higher levels, or more often, as noted in the previous examples.

### EXAMPLE 6

### NOS3 Polymorphism t-786c

The NOS3 gene codes for endothelial nitric oxide synthetase, which is a key enzyme in the production of nitric oxide (NO) to control vasodilation. The t-786c polymorphism in the NOS3 gene occurs 786 base pairs upstream of the start of the coding sequence in the promoter region, where a thymidine has been replaced by a cytosine. The variant polymorphism has been related to reduced transcription of the gene and reduced expression of NOS3. The only reported variant allele frequency is in African Americans of 5%, but we find the frequency of this allele in this group of cell lines to be 39%. The frequencies of the homozygous dominant, heterozygous and homozygous variant genotypes in the group of cell lines were 46%, 32% and 23% respectively.

The genotypes differed in their relative sensitivities (p<0.05, Friedman ANOVA), and the homozygous variant genotype was more sensitive than the homozygous dominant or heterozygous genotype to all the drugs (p<0.001, Tukey-Kramer test). The homozygous dominant and heterozygous genotypes did not differ in sensitivity. In the particular case of the drug thioguanine, the homozygous variant genotype reduced survival to 68% of the homozygous dominant genotype and 35% of the heterozygous genotype, and the difference among the genotypes was statistically significant (p=0.018, ANOVA). Overall, the homozygous variant genotype survival was 78% of the homozygous dominant, and 84% of the heterozygous genotype. The homozygous variant genotype was more sensitive to 80% of the drugs than the homozygous dominant and 70% of the drugs than the heterozygous genotype.

These unexpected findings demonstrate that the NOS3 homozygous variant genotype sensitizes cells to killing. Those people with the homozygous variant genotype would benefit from minimizing their exposure to toxic agents, and using products that counteract toxic damage at higher levels, or more often, as noted in the previous examples.

### EXAMPLE 7

### Delivery of DNA Repair Enzyme to Increase Repair of Cellular DNA

The purified Arabidopsis OGG1 DNA repair enzyme was encapsulated in pH sensitive liposomes, using liposomes for encapsulation of DNA repair enzymes as described in the '211 and '231 patents. The protein concentration inside the liposome was 100 μg/ml. Cultures of human keratinocyte line HaCaT cells were treated with 100 μM FeSO₄ and 100 μM CuSO₄ in aqueous buffer for 10 minutes then hydrogen peroxide was added to 500 μM for 10 minutes to produce oxidative damage, and particularly 8-oxo-guanine in the cellular DNA. After the FeSO₄/CuSO₄/H₂O₂ was removed, some of the cultures received the OGG1 encapsulated liposomes to a final concentration of 0.3 μg of liposomal OGG1 protein per ml of cell culture media. Control cultures received identical liposomes lacking any encapsulated protein. The cultures were either taken immediately, or incubated for 2, 6 or 24 hours. At the appropriate time, DNA was isolated from the cultures and analyzed for remaining 8-oxo-guanine using the endonuclease sensitive site assay and alkaline agarose gels as described in the '211 and '231 patents, except in this case the enzyme for detection of residual 8-oxo-guanine was OGG1.

The results are shown in Table 3. Treatment of cells with the hydrogen peroxide-iron and copper sulfate combination created about five 8-oxo-guanine bases per megabase of DNA. Under conditions of normal repair, about 60% of these were removed in 2 hours, leaving about 40%. However, in cells treated with liposomal OGG1, the 8-oxo-guanine damage was completely removed by 2 hours. The effect was due to the active enzyme, since empty liposomes added to cells resulted in about the same repair as observed with no liposomes.

These results demonstrate that repair which is not accomplished by the endogenous OGG1 repair enzyme can be completed by OGG1 enzyme added exogenously by liposome delivery. A product containing the liposomal form of the OGG1 enzyme would be particularly useful for those with the homozygous variant or heterozygous genotypes at the OGG1 S326C gene locus.

### EXAMPLE 8

### Correlation Between Genotype at the Gene Locus and Growth Inhibition of a Panel of Cell Lines

The foregoing results are summarized in Table 4 below, which expresses the relative resistance of the genotypes to toxicity, and is used to guide counseling on the relative benefit of each genotype to human health.

These results are expressed in greater detail, with data and results of statistical tests, in Table 5.

As can be appreciated from these tables, the health benefit of a gene is more complicated than the assumption that the homozygous dominant form is always better than the homozygous variant form. This finding, which is counter-intuitive, emphasizes the importance of the invention in screening groups of individuals and/or counseling individual people based on actual correlations between genotypes and sensitivity to toxic agents.

Although preferred and other embodiments of the invention have been described herein, further embodiments may be perceived by those skilled in the art without departing from the scope of the invention as defined by the following claims.

TABLE 1
NCI screening panel tumor cell lines and organ source

| Cell Line | Organ | Cell Line | Organ |
|---|---|---|---|
| NCI-H23 | Lung* | CCRF-CEM | Leukemia |
| NCI-H522 | Lung | K-562 | Leukemia |
| A549/ATCC | Lung | MOLT-4 | Leukemia |
| EKVX | Lung | HL-60(TB) | Leukemia |
| NCI-H226 | Lung | RPMI-8226 | Leukemia |
| NCI-H322M | Lung | SR | Leukemia |
| NCI-H460 | Lung | UO-31 | Kidney |
| HOP-62 | Lung | SN12C | Kidney |
| HOP-92 | Lung | A498 | Kidney |
| HT29 | Colon | CAKI-1 | Kidney |
| HCC-2998 | Colon | 786-0 | Kidney |
| HCT-116 | Colon | ACHN | Kidney |
| SW-620 | Colon | TK-10 | Kidney |
| COLO 205 | Colon | LOX IMVI | Melanoma |
| HCT-15 | Colon | MALME-3M | Melanoma |
| KM12 | Colon | SK-MEL-2 | Melanoma |
| MCF7 | Breast | SK-MEL-5 | Melanoma |
| NCI/ADR-RES | Breast | SK-MEL-28 | Melanoma |
| MDA-MB-231 | Breast | M14 | Melanoma |
| HS 578T | Breast | UACC-62 | Melanoma |
| MDA-MB-435 | Breast | UACC-257 | Melanoma |
| BT-549 | Breast | PC-3 | Prostate |
| OVCAR-3 | Ovarian | DU-145 | Prostate |
| OVCAR-4 | Ovarian | SNB-19 | CNS |
| OVCAR-5 | Ovarian | SNB-75 | CNS |
| OVCAR-8 | Ovarian | U251 | CNS |
| IGROV1 | Ovarian | SF-268 | CNS |
| SK-OV-3 | Ovarian | SF-295 | CNS |
| | | SF-539 | CNS |

*Non-small cell lung carcinoma

TABLE 2
Drugs with NSC number and Mechanism of Action Class

| NSC | Drug | Class |
|---|---|---|
| 750 | Busulfan | Alkylating |
| 762 | Nitrogen Mustard | Alkylating |
| 3088 | Chlorambucil | Alkylating |
| 6396 | Thiotepa | Alkylating |
| 8806 | Melphalan | Alkylating |
| 26980 | Mitomycin C | Alkylating |
| 34462 | Uracil N mustard | Alkylating |
| 79037 | CCNU | Alkylating |
| 95441 | MeCCNU | Alkylating |
| 95466 | PCNU | Alkylating |
| 119875 | cisPt | Alkylating |
| 172112 | Spirohydantoin Mustard* | Alkylating |
| 178248 | Chlorozotocin | Alkylating |
| 256927 | CHIP | Alkylating |
| 271674 | Carboxyppt | Alkylating |
| 338947 | Clomesone | Alkylating |
| 348948 | Cyclodisone | Alkylating |
| 353451 | Mitozolamide | Alkylating |
| 363812 | Tetraplatin* | Alkylating |
| 409962 | BCNU | Alkylating |
| 757 | Colchicine* | Anti-mitotic |
| 49842 | Vinblasine | Anti-mitotic |
| 67574 | Vincristine | Anti-mitotic |
| 125973 | Taxol | Anti-mitotic |
| 94600 | Camptothecin | Topo I inhibitor |
| 122819 | VM-26 | Topo II inhibitor |
| 123127 | Doxorubicin* | Topo II inhibitor |
| 141540 | VP-16 | Topo II inhibitor |
| 249992 | m-AMSA | Topo II inhibitor |
| 267469 | d-doxorubicin | Topo II inhibitor |
| 740 | Methotrexate | DNA/RNA |
| | | antimetabolite |
| 19893 | 5-fluorouracil | DNA/RNA |
| | | antimetabolite |
| 102816 | 5azaC | DNA/RNA |
| | | antimetabolite |
| 264880 | 5,6-d5azaC | DNA/RNA |
| | | antimetabolite |
| 752 | Thioguanine | DNA antimetabolite |
| 755 | Thiopurine | DNA antimetabolite |
| 27640 | 2′d5FU | DNA antimetabolite |
| 32065 | Hydroxyurea | DNA antimetabolite |
| 63878 | Ara-C | DNA antimetabolite |
| 303812 | Aphidicolin | DNA antimetabolite |

*not all cell lines were tested with these drugs

TABLE 3
DNA damage in HaCaT cells treated with
Hydrogen Peroxide/FeSO₄/CuSO₄ and OGG1 liposomes.

| | | 8-oxo- | Percent |
|---|---|---|---|
| Liposome | Time (h) | Gua/megabase | Remaining |
| None | 0 | 5.01 | 100 |
| None | 2 | 1.87 | 37 |
| None | 6 | 2.01 | 40 |
| OGG1 | 2 | 0 | 0 |
| OGG1 | 6 | 0 | 0 |
| Empty control | 2 | 1.67 | 33 |
| Empty control | 6 | 1.25 | 25 |

TABLE 4
Correlation of Genotypes with Resistance to Toxicity

| | Homozygous | | Homozygous |
|---|---|---|---|
| Gene Locus | dominant | Heterozygous | variant |
| TP53 P72R | Better | Worst | Best |
| OGG1 S326C | Better | Best | Worst |
| ERCC2 D312N | Best | Equal to homozygous | Equal to |
| | | variant | heterozygous |
| XRCC1 R194W | Equal to | Equal to homozygous | Not observed |
| | heterozygous | dominant | |
| XRCC1 R399Q | Worst | Better | Best |
| NOS3 t-786c | Equal to | Equal to homozygous | Worst |
| | heterozygous | dominant | |

TABLE 5
Detailed Correlation of Genotypes with Resistance to Toxicity

| | Relative Resistance (% sensitive)* | | | |
|---|---|---|---|---|
| | Variant/ | Variant/ | Hetero/ | ANOVA |
| Gene Locus | Dominant | Hetero | Dominant | p-va1ue** |
| TP53 P72R | 1.31 (43%) | 2.10 (13%)‡ | 0.73 (85%)‡ | <0.001 |
| OGG1 S326C | 0.86 (60%) | 0.76 (73%)‡ | 1.20 (25%)† | 0.0005 |
| ERCC2 D312N | 0.91† (70%) | 1.04 (45%) | 0.90 (83%)‡ | 0.0003 |
| XRCC1 R194W | {no variant | {no variant | 1.08 (53%) | 0.669*** |
| | alleles} | alleles} | | |
| XRCC1 R399Q | 1.53 (18%)‡ | 1.40 (35%) | 1.17 (28%)† | <0.0001 |
| NOS3 t-786c | 0.78 (80%)‡ | 0.84 (70%)‡ | 0.99 (68%) | <0.001 |

*GI₅₀ of numerator divided by GI₅₀ of denominator; value of <1 indicates greater sensitivity of numerator. In parenthesis is the percentage of drugs in which the numerator genotype was more sensitive than the denominator genotype.
**Friedman nonparametric ANOVA post-tests † p < 0.05, ‡ p < 0.01
***Wilcoxon matched-pairs signed-ranks test

1-4. (canceled)

5. A genetic counseling method comprising comparing a human subject's genotype at a gene locus with a correlation between genotypes at the gene locus and growth inhibition of a panel of cell lines, wherein:
(i) the gene locus exhibits at least two polymorphic allele forms;
(ii) the genomes of the cells lines of the panel comprise at least two genotypes at the gene locus; and
(iii) the correlation is obtained by a challenge of the panel with at least one toxic agent.

6-8. (canceled)

9. A method for genetic counseling of a human subject comprising comparing one or more polymorphisms in the subject's genotype with the relative sensitivity to toxic agents of cells with the same one or more polymorphisms compared to cells with different one or more polymorphisms.

10. The method of claim 5 further comprising using the comparison to advise the subject with regard to one or more therapeutic, nutritional, and/or cosmetic treatments.

11. The method of claim 10 wherein the one or more treatments comprise application of a topical formulation.

12. The method of claim 11 wherein the topical formulation comprises at least one DNA repair enzyme.

13. The method of claim 12 wherein the at least one DNA repair enzyme comprises T4 endonuclease V, photolyase, O⁶-alkylguanine-DNA alkyltransferase, and/or 8-oxo-guanine glycosylase.

14. The method of claim 12 wherein the at least one DNA repair enzyme is encapsulated in liposomes.

15. A kit for practicing the method of claim 5 said kit comprising instructional materials relating to the comparison between the subject's genotype at the gene locus and the correlation.

16-17. (canceled)

18. A genetic screening method comprising comparing individual genotypes exhibited by a group of human subjects at a gene locus with a correlation between genotypes at the gene locus and growth inhibition of a panel of cell lines, wherein:
(i) the gene locus exhibits at least two polymorphic allele forms;
(ii) the genomes of the cells lines of the panel comprise at least two genotypes at the gene locus; and
(iii) the correlation is obtained by a challenge of the panel with at least one toxic agent.

19-22. (canceled)

23. The method of claim 18 wherein at least one member of the group is counseled based on the results of the comparison.

24. The method of claim 23 wherein the counseling comprises advising the at least one member of the group with regard to one or more therapeutic, nutritional, and/or cosmetic treatments.

25. The method of claim 24 wherein the one or more treatments comprise application of a topical formulation.

26. The method of claim 25 wherein the topical formulation comprises at least one DNA repair enzyme.

27. The method of claim 26 wherein the at least one DNA repair enzyme comprises T4 endonuclease V, photolyase, O⁶-alkylguanine-DNA alkyltransferase, and/or 8-oxo-guanine glycosylase.

28. The method of claim 26 wherein the at least one DNA repair enzyme is encapsulated in liposomes.

29. The method of claim 18 wherein the group is representative of all humans or is selected on the basis of ethnicity, race, religion, geographic region, and/or common hereditary descent.

30-45. (canceled)

46. A method for obtaining a correlation between polymorphic allele forms at at least one gene locus and sensitivity of humans to toxic agents comprising:
(A) selecting a panel of cell lines where the genotypes of the panel comprise at least two of said polymorphic allele forms at said at least one gene locus;
(B) determining the relative sensitivity of each cell line to growth inhibition produced by one or more toxic agents; and
(C) correlating said relative growth inhibition of each cell line with its genotype at the said locus.

47-66. (canceled)

67. The method of claim 9 further comprising using the comparison to advise the subject with regard to one or more therapeutic, nutritional, and/or cosmetic treatments.

68. The method of claim 67 wherein the one or more treatments comprise application of a topical formulation.

69. The method of claim 68 wherein the topical formulation comprises at least one DNA repair enzyme.

70. The method of claim 69 wherein the at least one DNA repair enzyme comprises T4 endonuclease V, photolyase, O⁶-alkylguanine-DNA alkyltransferase, and/or 8-oxo-guanine glycosylase.

71. The method of claim 69 wherein the at least one DNA repair enzyme is encapsulated in liposomes.

72. A kit for practicing the method of claim 9 said kit comprising instructional materials relating to the comparison between the subject's genotype at the gene locus and the correlation.
